(19) United States
(12) Patent Application Publication
(10) Pub. No.: US 2014/0308727 A1
(43) Pub. Date: Oct. 16, 2014

(54) Anaerobic Digestion System

(71) Applicant: Luigi Castelli, Udine (IT)

(72) Inventor: Luigi Castelli, Udine (IT)

(21) Appl. No.: 14/246,341

(22) Filed: Apr. 7, 2014

Related U.S. Application Data
(60) Provisional application No. 61/812,294, filed on Apr. 16, 2013.

(51) Int. Cl. C12P 5/02

(52) U.S. Cl. CPC C12P 5/023
USPC 435/167; 435/294.1

(57) ABSTRACT

An anaerobic digestion system for digestion of animal or other organic material and production of biogas is shown. The digestion system includes a simplified construction and improved agitation system both partially enabled by a dual container system that makes use of the buoyancy of the digestion container for support.

### CROSS-REFERENCE TO RELATED APPLICATIONS

This application claims priority to U.S. Provisional application No. 61/812,294 titled: Anaerobic Digestion System for Production of Biogas from Agricultural Biomass, Animal Manure and Waste Water and Organic Solid Waste, filed on 16 Apr. 2013 by the same inventor and currently pending.

### BACKGROUND OF THE INVENTION

1. Technical Field

This invention is relative to an anaerobic digestion system for the production of biogas from various organic matters, such as agricultural biomass, sludge and solid waste.

2. Related Background Art

The anaerobic treatment of biomass and wastes has recently become very important, because it generates energy without negative environmental impact. Biogas is a very valuable form of energy, because it contains methane, a combustible gas which during combustion produces two chemical compounds which are harmless for the environment: water and carbon dioxide from a renewable source.

It is therefore understandable how the production of biogas in specialized plants is a valid tool in the fight against global warming caused by the accumulation in the atmosphere of carbon dioxide produced by the use of fossil fuels and by uncontrolled fermentation of organic wastes. In addition, biogas use is extremely practical and versatile, as a matter of fact it can be used directly in internal combustion engines and gas turbines for the co-generation of electric energy and heat or, after an adequate refining treatment, it can be piped to the distribution grids of fossil methane. Methane gas can be compressed and stored in cylinders and used to fuel gas-propelled vehicles. This application appears particularly interesting for biogas produced from municipal solid waste because the availability of this organic matter occurs in the urban areas, exactly where a non-polluting fuel is particularly needed.

An additional advantage of biogas production plants is the opportunity of treating the anaerobically digested output material to produce an organic fertilizer (compost), which can be used in agriculture. Of course, in order for compost to be used in the food chain, toxic compounds must not be included in the input material of the anaerobic digestion system.

In the current technology, the use of horizontal cylindrical digesters for biogas production, with batch or continuous process, is known. For instance, the patent EP 0476 217 describes a horizontal cylindrical digester which is heated through hot water circulation inside vanes of the shell, while the patent EP 0374708 describes a horizontal cylindrical digester immersed in a heating liquid.

The system described in EP 0476217 is provided with an agitation system for the treated material, which, in addition to being very complex from the construction point of view, has high electricity consumption and requires heavy maintenance. These problems are present also in the system described in EP 0374708, which uses a rotating internal auger to move the material toward the outlet of the digester.

In another known system, called “garage-type” such as that described in patent WO 02/31104, heat is transferred by the percolation liquid (heated in dedicated heat exchangers) and by the surface of the heated floor. It is evident that the heat transfer through the walls of the “garage” is not very efficiency due to the poor contact. Because of these limitations, all garage-type systems operate with a mesophilic process at a temperature of approximately 35-40° C., while the thermophilic systems, which use bacteria developing at a temperature of approximately 55° C., have a superior biogas yield and provide a more efficient hygienisation. Another disadvantage of this system is its inability to process material with a low porosity. In fact, it is necessary to treat the material by mixing it with structural material, such as wood chips, in order to improve the porosity of the mass.

The process described in U.S. Pat. No. 4,735,724 uses a non-mixed vertical digester, designed to concentrate the solid matter in the upper part, while the digested material is removed from the bottom. This system has operational problems and difficulties during the phase of replacing the material to be treated.

Another known system is described in patent US 2002/0192810, where the reactor is horizontal, rotating a cylinder submersed in a liquid and used for the aerobic composting of sludge which has been already anaerobically digested in a particular U-shaped digester. This type of system is not designed for anaerobic digestion; in fact it is provided with ducts for the entrance of the air required by the composting process. In addition the rotation system is complex and expensive, which heavily impacts on the construction and amortisation costs. Even more complex and onerous from the construction and operation points of view is the system described in U.S. Pat. No. 5,427,947, which includes a rotating cylindrical reactor for composting.

Other systems are known, where the digestion includes more phases of biological treatment, such as the two-phase system described in patent U.S. Pat. No. 6,342,378 and the three-phase system of U.S. Pat. No. 5,269,634, which uses three material containing reactors, having different treatment times, interconnected by a piping system suitable for pumping the process liquid from a reactor to another.

The two systems described in the French patent 2772641 and in US 2011/0256603 are provided with a rotating cylindrical digester immersed in a liquid, but neither system is able to process non-pumpable material having a high content of dry matter. In all of the systems there are internal structures to facilitate mixing. For this reason, all of the above mentioned known systems cannot be push-loaded with material packaged in roto-bales or bales, or with untreated municipal solid waste, while the system of the present invention allows such. The present invention overcomes the mentioned disadvantages and inconveniences, with the objective of providing a new system characterized by design simplicity and low construction and operation costs.

### DISCLOSURE OF THE INVENTION

The object of this invention is an anaerobic digestion system for the production of biogas from agricultural biomass, animal manure and waste water and organic solid waste, consisting substantially of one or more horizontal digesters, installed in parallel or adjacent to each other, immersed in a heated liquid, generally water.

The digesters are shaped as cylinders, with their ends fixed to the opposite walls of the tank containing the heated liquid. In another embodiment, horizontal, cylindrical digesters float in the liquid contained by the tank without constraining their horizontal axis in the vertical plane, with subsequent drastic reduction of vertical loads applied to the digesters during the phases of loading and unloading and possibility of eliminating the rotation supports. Due to the absence of constraints in the vertical plane, the digesters can freely float and the weight difference is automatically compensated during the loading and unloading phases.

This embodiment allows for building the digester with a light structure, which must resist only loads uniformly distributed on an ample surface. The system with the digesters fixed to the walls of the tank is suitable for batch processing materials having high dry matter content which are push-loaded through the loading opening located on the tank wall. The digested material is unloaded through the opposite opening of the digester, from where it is pushed out by the fresh material loaded at the opposite side of the digester.

Fresh material consisting of agricultural biomass or waste packaged in bales, preferably roto-bales of cylindrical shape, is loaded in a very simple manner by a fork lift truck or by an agricultural tractor provided with lifting forks. To allow the loading operation, the roto-bales have a diameter which is smaller than the inside diameter of the digester. The difference in diameter also facilitates the internal circulation of the inoculation liquid and its adsorption by the roto-bales. Bulk materials are loaded by a special push-loader, which can translate on a trolley to position itself just in front of the loading mouth of each digester.

Once the digester has been loaded with fresh material, and at the same time the digested material has been unloaded from the digester, the two ends of the digesters are closed by means of covers which are gas and water-tight. The biological process of digestion is initiated by filling the digester, partially or totally, with liquid derived from a former digestion treatment, such as that resulting from the dewatering of the digested material, or with animal waste water. It is also possible to initiate the process through the partial recirculation of pre-digested solid material. The biogas produced inside the digester is collected by take-off ports located in the upper part of the digester and conveyed to an accumulation system (gas holder). The biogas is taken from the gas storage to be used, after dehumidification and desulphurization, in electric power generators, or to be sent to a refining system able to adsorb the carbon dioxide and increase the methane content before the inlet to the grid or the compression into cylinders.

At the end of the digestion process, which lasts for approximately two or three weeks, the liquid is drained from the digester and collected in a tank. The liquid drainage takes place through a set of drains located in the lower part of the digester or of the end covers, preferably on the outlet cover.

Once the drainage of the liquid from the digester is completed, the outlet cover is opened. At the outlet end of the digester there is an open tank (basin) for the collection of the solid digested material and liquid remained inside the digester. The collection tank is provided with a drainage system for the liquid, which is taken to a container, where it waits to be recycled to the digester for a new cycle or to be recovered in another manner. The solid material accumulated in the tank is removed by means of a wheel loader and used according to the programmed scheme, recirculation to the digester, dewatering, composting or spreading on agricultural fields. When the liquid contained by the digester has been drained, the inlet cover is also opened.

The heating and hygienisation of the fresh material loaded into the digester take place by heat transfer from the liquid contained in the tank containing the digester through the digester wall. The tank can be built of reinforced concrete or any other water-proof material, while the cylindrical shell of the digester, fixed to the tank bottom with anti-floating tension rods, is made of corrosion resistant materials, such as plastic, fibreglass reinforced plastic or stainless steel. Heat is transferred from the tank liquid to the processed material through the digester wall, which therefore is not insulated. Instead, the tank walls may be insulated as well as the two end covers of the digesters.

The heat required for heating the liquid of the tank may be provided by the biogas fuelled cogeneration unit, or by solar radiation of the tank, which in this case is provided with a transparent enclosure in order to obtain the greenhouse effect and, at the same time, reduce the heat loss through the free surface of the liquid. The system is completed with the containers for the inoculation liquid, animal waste water and/or percolate and discharged liquid, and by the system of pumps, valves and piping required to convey this liquid to the digester and to discharge it. The liquid charging and discharging is done though openings and drains located on the digester wall or on the outlet cover. In any case, there are grates to prevent the formation of occlusions in the liquid drains. The loading cover is opened only after the liquid has been completely drained from the opposite opening. The digester is slightly inclined toward the unloading side to favor the draining.

According to this invention, the system is completed by the equipment required for filling the tank containing the heating liquid and by the relative heating systems, such as co-generator, boiler, heat exchanger, pumps, valves, etc. When the system processes material, which cannot be pumped, the batch process may also include two or more phases. The treatments carried out inside the digesters may be all biological, hydrolysis and bio-methanisation, or a chemical pre-treatment of hydrolysis may take place, which results particularly useful in the digestion of agricultural wastes, such as rice straw. The system described above is suitable for the treatment of material having a high dry matter content, while the variant design with floating digester can accept as input only pumpable material, therefore when organic waste is treated it is necessary to shred and pulp it. The floating digester can easily rotate around its axis, driven by two pairs of motorized wheels contacting the upper surface of the digester, or the rotation may be provided by a belt or rope system encircling the digester and activated by two rotating winding drums. In the design with winding belt, the rotation necessarily alternates in opposite directions. Depending on the process requirements, the rotation may be continuous or intermittent.

Other embodiments include methods for using the floating digesters. In one embodiment, the liquid is transferred from one digester to another using a series, parallel or series/parallel combined scheme. This embodiment includes the pumps, piping and valves necessary for operation. To allow for the vertical movement of the digester during the loading and unloading operations, flexible pipes are used, which are supported by adequate guiding means. The same pipes can follow the digester during its alternate rotation, if provided, while in the case of mono-directional rotation, fast-coupling pipes are preferred, which are disconnected once the unloading and loading operations are completed.

In another embodiment of this invention, to prevent the introduction of oxygen into the digester, the draining of the digested liquid can take place by pressurising the digester with biogas or inert gas in order to obtain the complete unloading of the fluid from one or more drains located in the bottom part of the digester or on its end covers. In addition, the digester can be easily inspected and its inside can be easily cleaned after having removed the end covers. Finally, according to another feature of the invention, the liquid containing tank contains floating elements, such as hollow plastic spheres or items made of polystyrene foam, with the purpose of limiting the heat loss through the free surface of the liquid.

The present invention is particularly advantageous when the required production capacity is not high and other known systems are not economically feasible, as in the case of small farming operations, or when it is intended to produce biogas from the organic waste generated by small communities.

### BRIEF DESCRIPTION OF THE DRAWINGS

These and other characteristics of this invention will appear evident from the following description, which without limitations is a preferred example of design, and in the attached drawing sheets, where:

FIG. 1 shows a longitudinal section view of a cylindrical digester 1 fixed in the structure of the tank and immersed in the heated liquid;

FIG. 2 shows a plan view of a system including four parallel digesters immersed in the same tank containing heated liquid;

FIG. 3 shows a lateral view of the loading side of the system illustrated in the previous figure;

FIG. 4 shows a partial longitudinal section view of a digester during the phase of roto-bales loading by means of a specific fork lift truck;

FIG. 5 shows a partial longitudinal section view of a digester fixed to the tank with a loading/unloading push-system for bulk materials;

FIG. 6 shows a partial longitudinal section view of the previous figure during the following phase of pushing the bulk material inside the digester;

FIG. 7 shows a front view of the cylindrical digester of the trolley for bulk material loading with its push equipment;

FIG. 8 shows a front view of the tank with the four cylindrical digesters and gives evidence to the top transparent coverings used for achieving the greenhouse effect;

FIG. 9 shows a longitudinal section view of a floating cylindrical digester for treatment of fluids; it is shown empty, thus it floats and gets over the upper edge of the tank;

FIG. 10 shows a front view of the previous figure;

FIG. 11 shows a longitudinal section view of a cylindrical digester which is partially immersed because part of it has been filled with the fluid to be processed;

FIG. 12 shows a front view of the previous figure;

FIG. 13 shows a longitudinal section view of the same digester of the previous four figures, fully immersed in the liquid of the tank because totally filled with the fluid to be processed;

FIG. 14 shows a front view of the previous figure;

FIG. 15 shows a longitudinal section view of a floating cylindrical digester equipped with a belt device which partially winds the external lateral surface and enables the rotation of the digesters (around its longitudinal axis) in the liquid of the tank. Here it is shown empty and consequently it floats in the liquid;

FIG. 16 shows a front view of the previous figure, and gives evidence to the two belt winding units;

FIG. 17 shows a longitudinal section view of the same digester of the two previous figures, partly filled with the fluid to be processed and consequently it is partially immersed in the liquid of the tank;

FIG. 18 shows a front view of the previous figure;

FIG. 19 shows a longitudinal section view of the same digester of the four previous figures, but it is fully filled and immersed in the liquid of the tank, with the belt system designed to allow for the rotation of the digester around its longitudinal axis;

FIG. 20 shows a front view of the previous figure.

FIG. 21 shows an embodiment including an agitation system.

### DETAILED DESCRIPTION

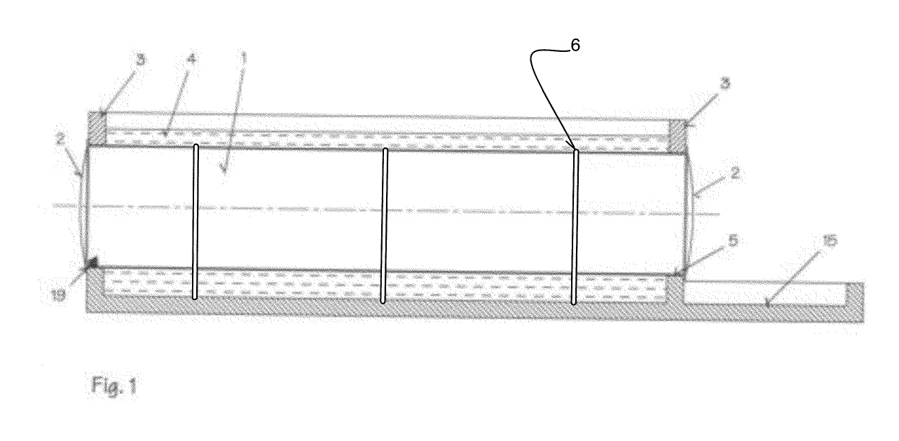

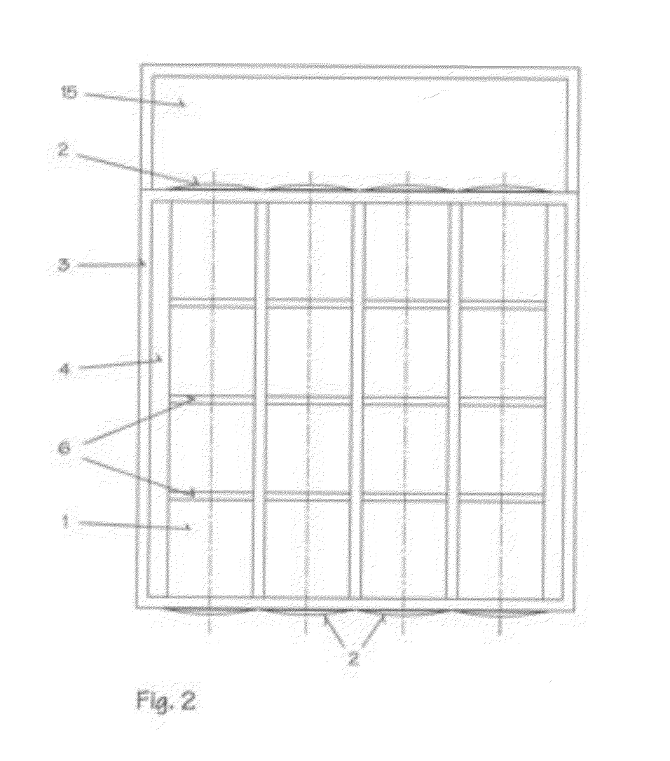

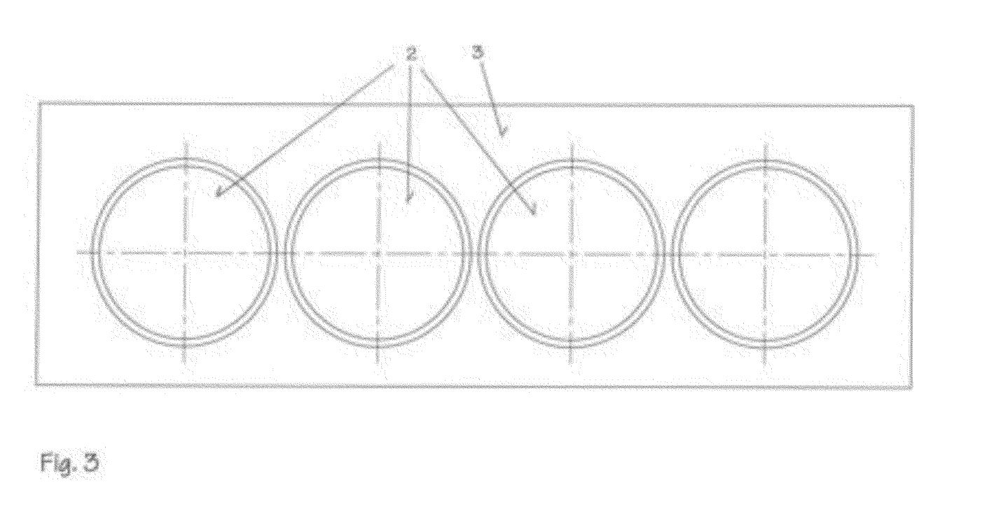

As it can be seen in the attached figures, this invention is relative to a new modular system of anaerobic digestion, substantially characterised by one or more horizontal digesters immersed in a heated liquid. In the first example shown in FIGS. 1-3, four parallel digesters are installed side by side. Each digester consists substantially of a cylindrical container 1, with its two ends closed by detachable covers 2, which is fixed to the two opposite walls of the tank 3 containing the heated liquid 4. The digester is gas and water-tight and it is provided with adequate seals 5 built during the construction phase to prevent the leakage of the liquid from the tank. In addition, these digesters, being immersed in the liquid, are subject to buoyancy, particularly when they are empty or during the filling phase, thus various tension rods 6 encircle the lateral surface of the digester and are anchored to the tank floor. In another embodiment not shown the digesters are parallelepipeds.

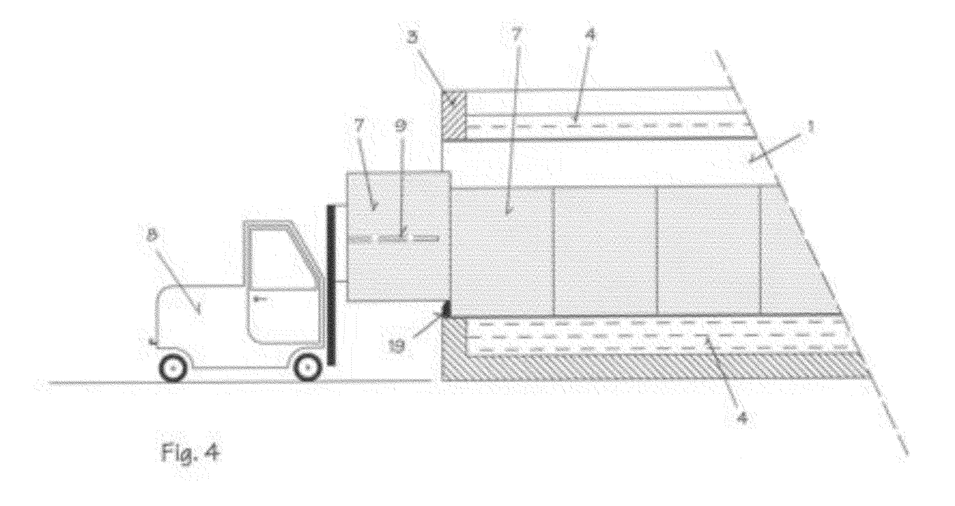

The present digester can be used to anaerobically treat both materials which can be pumped (wet digestion) and bulk material (dry digestion). Materials that can be pumped enter the digester through one or more openings of the shell or covers (not shown) and are unloaded through openings built in the lower part of the digester (not shown.) In the case of treatment of materials which can be pumped, the digester can also be emptied through pressurisation with compressed gas, preferably biogas, and opening of one or more discharge ports. When materials which cannot be pumped are treated, the loading and unloading of the digester 1 occurs through a push-system. In a first case shown in FIG. 4, the solid material is packaged in roto-bales 7 and a fork lift truck 8 provided with forks 9 lifts each roto-bale cylinder and pushes it inside the digester. Of course, the cover at the other side of the digester must be open, otherwise the loading with new material is not possible. The roto-baled material slides over a tooth 19, in the lower part of the inlet opening so that the last loaded bale cannot slide back during the withdrawing of the forks. The material treated inside the digester is pushed out by the loading operation, and falls into a shallow external collection basin 15, from which the collected material is removed. The size of the hopper and the digester are selected to snugly fit the size of the roto-bales.

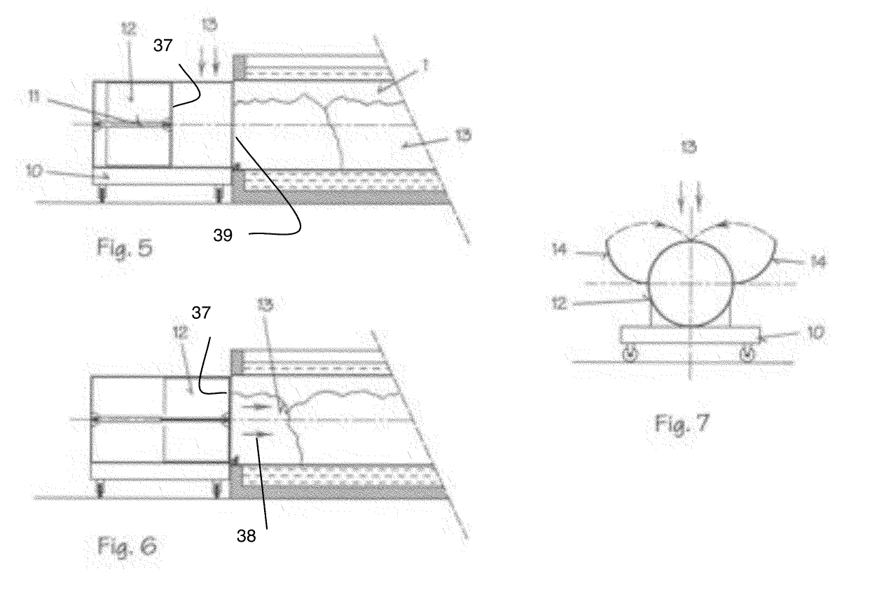

FIGS. 5-7 show an embodiment using a loading machine 11 for top loading of the bulk material. The loading machine is comprised of a circular cylinder 12 that can be aligned with the cylinder of the digester 1. The loading machine further includes a piston 37 fit in the interior of the cylinder 12 such that when the cylinder moves in the direction 38 as shown, the bulk material 13 is forced into the digester. The top of the loading machine 11, where the bulk material 13 is loaded, includes two half-covers 14, shaped as circular arcs, which close and latch to form a channel during the insertion phase and open for loading of bulk material. The loading machine 11 can move among multiple digesters by means of a trolley 10 to move the machine in alignment with the loading mouth of each digester. Once the insertion stroke has been completed, the piston is retracted to enable the compression chamber to be reloaded.

Another embodiment includes a method of using the digester 1 in conjunction with the loading machine 11. The end hatches of the digester are opened or removed and the loading machine is aligned with a first end of he digester. The doors 14 are opened and the loading machine is filled with bulk material. The doors 14 are then closed and latched and the piston 12 moves in the direction 38 to force the bulk material into the first end of the digester 39. Digested material already in the digester is simultaneously forced out the opposite end of the digester (not shown). Once the loading of the digester with fresh material and the simultaneous unloading of the digestate are completed, the two ends of the digesters are closed by means of the gas and liquid-tight covers (item 2 FIG. 1). Then the biological process is initiated by filling the digester, partially or totally, with liquid derived from a previous digestion process, or with animal waste water to be used for the production of biogas. The inlet of the liquid occurs through ports in the shell or in the covers with adequate means known in the industry. During the inlet of the liquid, air goes out through a vent valve, which is opened during this phase. In another embodiment the digestion is initiated through recirculation of digested material, which is mixed with the fresh organic bulk material 13 prior to its loading into the digester.

After completion of the digestion process, the liquid is discharged from the digester and collected in its tank. The liquid is discharged through a set of ports with valves, which are located in the bottom part of the digester or on the outlet cover, which is thus removed for discharging the material in the external collection basin 15.

The heating and hygienization of the fresh material take place through heat transfer between the digester wall and the liquid 4 contained by the tank 3 which contains the digester. Both tanks 3 and 15 can be made of reinforced concrete or other water-proof material, while the cylindrical shell 1, fixed to the tank bottom with anti-floating tension rods, is made of corrosion resistant materials, such as plastic, fibreglass reinforced plastic or stainless steel. The heat transfer from the tank hot liquid to the processed material occurs through the digester wall, which therefore is not insulated. The walls and bottom of the tank and the digester end covers, which are not in contact with the heating liquid, may be insulated. The heat required for heating the tank liquid may conveniently be provided by the biogas fuelled cogeneration unit. As shown in FIG. 8, the tank can be provided with a transparent cover in order to obtain the heating benefit of solar radiation.

The system is completed with a tank for the inoculation liquid (animal waste water and/or percolation liquid) and with the system of tanks, pumps, valves and piping necessary to convey this liquid to/from the digester. The pre-heating of the inoculation liquid is generally not required due to the excellent efficiency of the tank liquid in heating the digester.

The digester 1 must be heated in order to work efficiently and this function is performed by the liquid of the tank where the digester is installed. This liquid 4 may be heated by various known means, such as boilers and burners, operating also with the biogas produced by the digester. To keep the desired temperature of such liquid, the containing tank 3 can be provided with a roof system 20, preferably made of transparent material in order to add heat from solar radiation. The described digester is particularly suitable for processing material having a high dry matter content, while the variant with floating digester can be set up to either receive only materials which can be pumped, thus when organic solid waste is processed it is necessary to shred and pulp it or with the appropriate support system can be further attached to a loading machine.

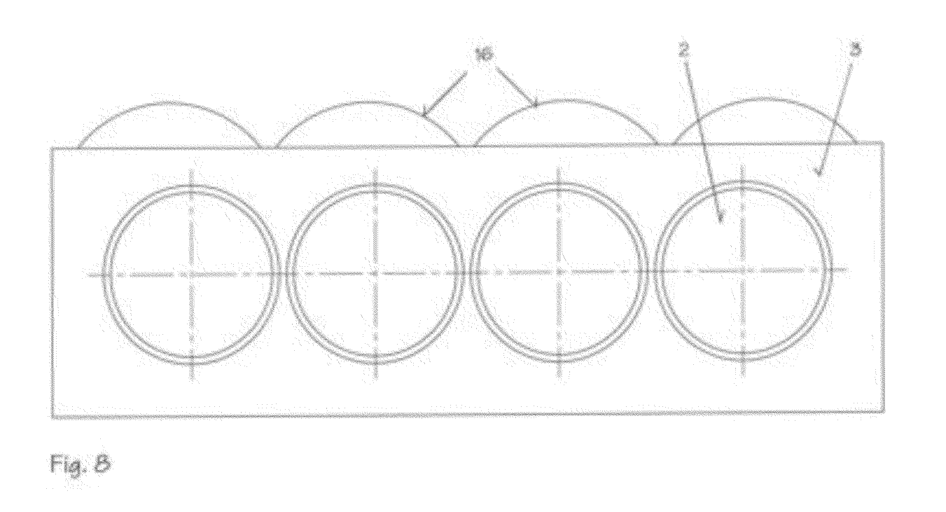

In another embodiment shown in FIGS. 9 to 20, the digester, which is still cylinder-shaped, floats in the heated liquid contained by the tank 3, where the buoyancy is not prevented by the tension rods of the previous embodiments, and thus the thickness of the digester shell can be even smaller, with subsequent saving in the construction cost. The floating digester 16 requires only to be guided during its vertical movements, which are shown in FIGS. 9-14. FIGS. 9-10 show an empty digester, FIGS. 11-12 show the digester during loading/unloading phases, and FIGS. 13-14 show a full digester. In addition, flexible piping (not shown) is provided for the digester loading/unloading operations.

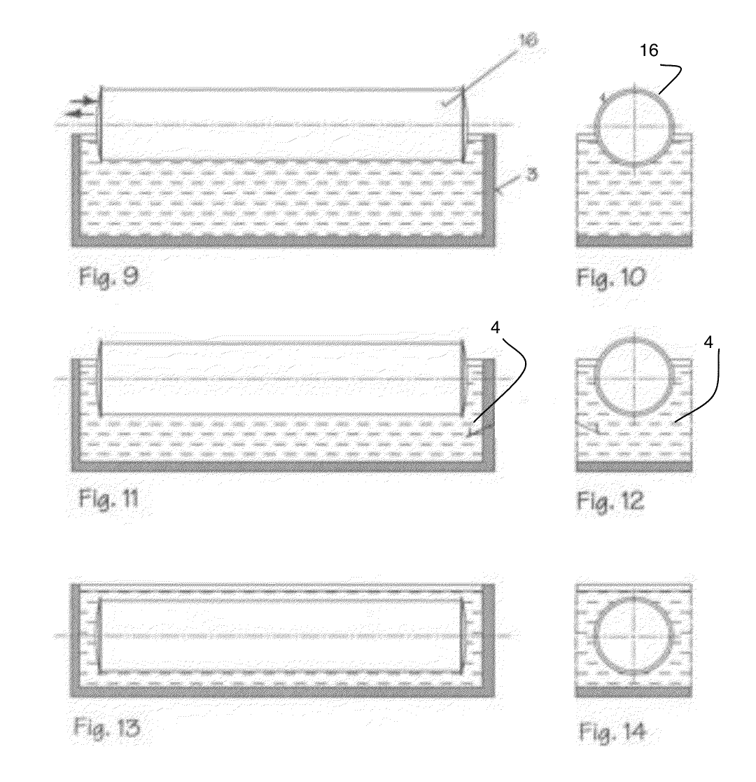

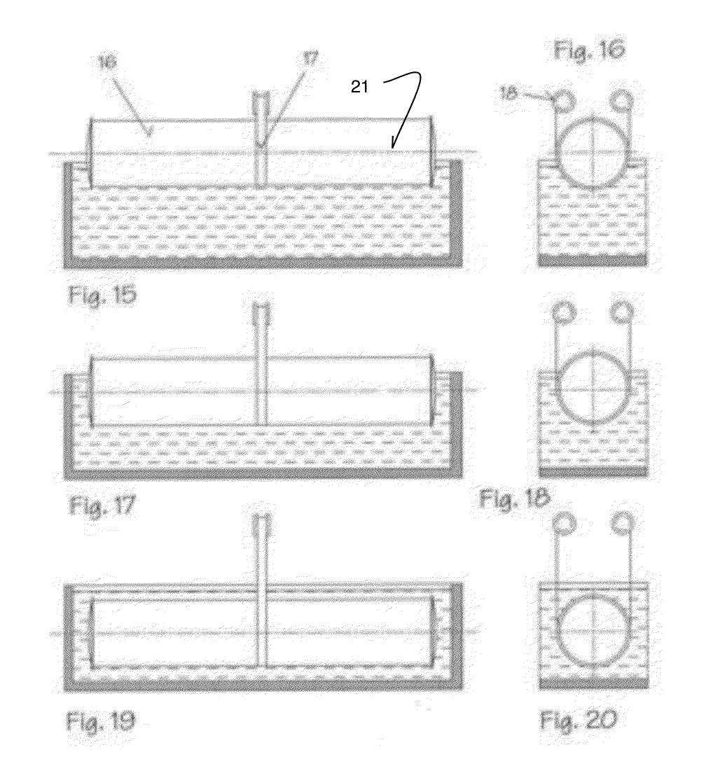

For stirring the contained material, the floating digester 16 of this invention can rotate around its longitudinal axis 21, driven by two pairs of top wheels, or as shown in FIG. 15-20 by means of devices with belt 17 wound on motorized rotating drums 18. In the design with belt, or rope, driving, the rotation is necessarily in alternate directions. Depending on the process requirements, the rotation of the digester can be continuous or intermittent. Compared with the known agitated systems, the digester of this invention exhibits the advantaged that the agitation system is not installed inside the digester, a location which makes maintenance very difficult, because it is necessary to empty completely the digester before getting access to the agitator. The floating digesters can be arranged in alternate system configurations and the liquid contained by the digesters can be transferred from one digester to another according to a series, parallel or combined series/parallel scheme. To allow for the vertical movements of the digester during the loading/unloading operations, adequate flexible pipes with guiding devices are used. The rotation rate of the digester is limited to prevent the loss of organic matter during the operating mode with continuous process, however the rotation is also useful to favor the gas escaping from the bath and to prevent the settling of solids in the digester bottom.

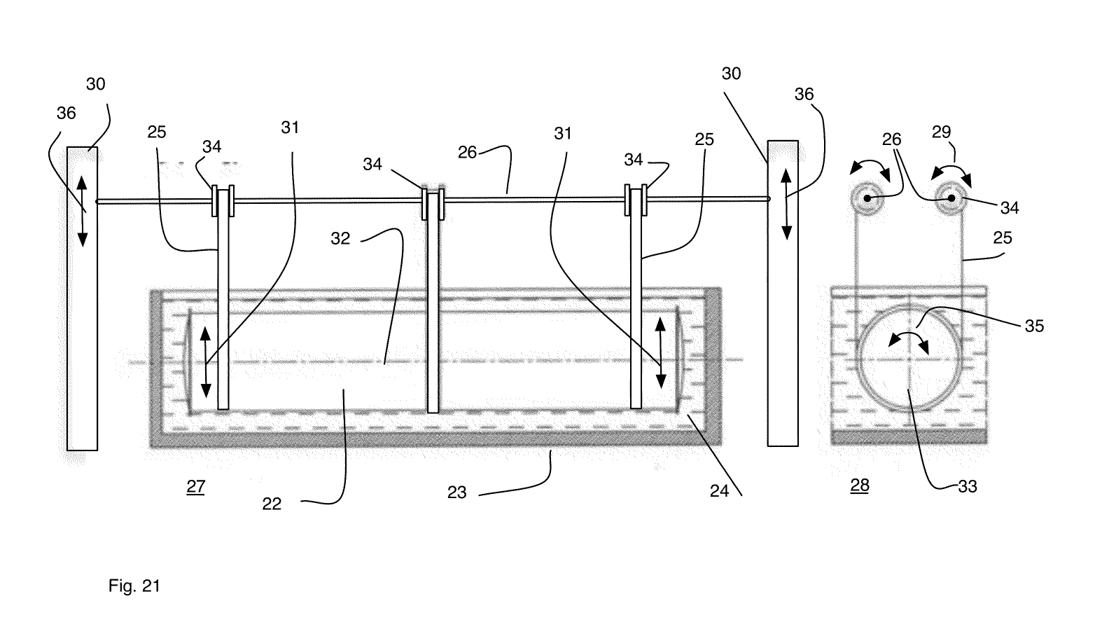

Another embodiment shown in FIG. 21 includes an agitation system with the floating tank design. A cross-sectional side view 27 and an end view 28 are shown. The embodiment includes a cylindrical tank 22 having a long axis 32 and a transverse vertical axis 33. The tank is located within a second enclosure 23 and the second enclosure is at least partially filled with fluid 24. The fluid helps to support the weight of the tank 22 through buoyancy. In the preferred mode the fluid is water. The tank is further contained and supported by a plurality of flexible straps 25. In the example of FIG. 21 three straps are shown, but more or fewer straps could be used. The straps loop around the tank 22 and ends of the straps are attached to rotatable pulleys 34. In one embodiment the pulleys are motorized such that they may be rotated about a central axis producing the motion 29. Motion 29 of the pulleys produces the rotational motion 33 of the tank about its long axis 32. The pulleys 34 may be rotated back and forth producing a rocking motion of the tank. The pulleys are further supported on a shaft 26 that is attached at its ends to supports 30. The supports include the ability to move the ends of the shafts vertically 36. In a preferred embodiment the ends may be moved independently such that each end of the tank 22 may be raised and lowered independently thereby producing a sloshing mixing of the contents of the tank 22. It is seen that the design allows mixing by rotation 35 about the long axis 32 by rotation of the pulleys 29 and mixing by tilting along the long axis of the tank by raising and lower each end of the support shaft 26 using the supports 30. Immersion of the tank 22 in the fluid 24 results in a buoyant support of the tank body to reduce the stress on the tank structure produces by mixing motions along multiple axes.

In another embodiment not shown the supports 30 are sufficiently tall to allow the tank 22 to be raised completely from the enclosure 23 such that the loading capabilities as described in FIGS. 4-7 are combined with the agitation capabilities as described in FIG. 21.

### SUMMARY

An anaerobic digestion system for digestion of animal or other organic material and production of biogas is shown. The digestion system includes a simplified construction and improved agitation system both partially enabled by a dual container system that makes use of the buoyancy of the digestion container for support.

What is claimed is:

1. Anaerobic digestion system for biogas production from organic materials using digesting fluids, comprising:
a. a tank having at least front and rear walls and containing a liquid;
b. means for the heating of the liquid contained by the tank;
c. one or more anaerobic digesters mounted within said tank and immersed in said liquid, said digesters each comprising:
i. a waterproof container having a cross-sectional shape, a long axis and a short axis, said container having a front open end and a rear open end, said open ends at each end of the long axis and said container long axis oriented in horizontal position within said tank wherein front and rear open ends of said container extend through and are sealed to holes in the front and rear walls of said tank;
ii. covers removably attached to the container at the front and rear open ends to provide gas-tight and liquid-tight seals at the open ends,
iii. ports for the loading and unloading of digesting fluids and evacuation of the biogas generated inside the digesters,
d. wherein the one or more digesters may be re-loaded with organic material by loading bales of the organic material into the front open end thereby pushing already digested organic material out the rear open end.

2. The digestion system of claim 1 wherein the cross-sectional shape is circular and the bales of the organic material are roto-bales.

3. The digestion system of claim 1 further comprising a hook located at the front open end and oriented such that as bales of the organic material are loaded into the front open end the bales slide over the hook and the hook engages the bales to prevent their movement out the front open end.

4. The digestion system of claim 1 further comprising a loading machine for loading bulk solid material into the digesters, said loading machine comprising:
i. a hopper, said hopper having a top, two ends and a cross-sectional shape and a size said cross-sectional shape the same as the cross-sectional shape of the digester and said size selected such that the hopper is capable of being fitted to mate with the front open end of the digester,
ii. a piston located within the hopper said piston having a cross-sectional shape and size, said cross-sectional shape the same as the hopper and said piston movable between a first position and a second position,
iii. doors attached to the top of the hopper,
iv. wherein when the loading machine is mated to the digester, the doors on the hopper may be opened and the piston located in the first position, the hopper may be loaded with the bulk solid material and the doors may be closed and the piston moved to a second position thereby forcing the bulk material into the front open end of the digester,
b. ports for the loading and unloading of digesting fluids and evacuation of the biogas generated inside the digesters.

5. The digestion system of claim 4 wherein the cross-sectional shape and size of the digester and the hopper are selected such that roto-bales snugly fit within the hopper and the digester.

6. An anaerobic digestion system for biogas production from organic materials using digesting fluids, comprising:
a. a tank having at least front and rear walls and containing a liquid;
b. means for the heating of the liquid contained by the tank;
c. one or more anaerobic digesters mounted within said tank and immersed in said liquid and at least partially supported by buoyancy, said digesters each comprising:
i. a waterproof container having a cross-sectional shape and a long axis, said container having a front open end and a rear open end and said container oriented with the long axis in horizontal position within said tank;
ii. covers removably attached to the container at the front and rear open ends to provide gas-tight and liquid-tight seals at the open ends,
d. a plurality of belts supporting the digesters, the belts having two ends, the ends attached to a plurality of pulleys, said pulleys capable of rotation such that when the pulleys rotate the belts transfer the rotary motion to the digesters causing the digesters to rotate about their long axis,
e. a shaft on which the pulleys are mounted the shaft having two ends,
f. a support for the shaft wherein the ends of the shaft are mounted in the support,
g. ports for the loading and unloading of digesting fluids and evacuation of the biogas generated inside the digesters.

7. The digestion system of claim 6 wherein the cross-sectional shape of the digester is circular.

8. The digestion system of claim 6 wherein the supports are motorized to raise and lower the two ends of the shaft independently such that when one end is raised and the other lowered the digester is tilted along its long axis

9. The digestion system of claim 8 wherein the pulleys and the supports may be activated simultaneously thereby producing a simultaneous rotary motion and tilting motion of the digester.

10. A method for digesting material for the production of biogas said method comprising:
a. loading the material into a hopper said hopper having a cross-section and a piston,
b. mating the hopper with a digester, said digester comprising a container having a first end and a second end, said ends removably sealed by covers, said container having the same cross-section as the hopper,
c. pushing the material into the digester through an opened first end of the digester and thereby forcing an already digested material out an opened second end of the digester,
d. unmating the hopper from the digester,
e. sealing the first end and the second end of the digester,
f. adding a digestion starting material to the digester,
g. immersing the digester in a fluid filled tank such that the fluid partially supports the weight of the digester through buoyancy,
h. heating the fluid to maintain a minimum temperature in the digester,
i. attaching belts around the digester, said belts having two ends attached to rotatable pulleys said pulleys attached to a shaft, said shaft having two ends and said ends of the shaft attached to a support and rotating the pulleys , thereby causing the digester to rotate,
j. removing biogas generated from the generator.

11. The method of claim 10 wherein the supports are motorized to raise and lower the two ends of the shaft independently such that when one end is raised and the other lowered the digester is tilted along its long axis and further including raising and lower the supports.

12. The method of claim 11 further including raising and lowering the supports while simultaneously rotating the pulleys thereby producing a simultaneous rotary motion and tilting motion of the digester.
